(19) United States
(12) Patent Application Publication
(10) Pub. No.: US 2011/0044991 A1
(43) Pub. Date: Feb. 24, 2011

(54) METHODS AND COMPOSITIONS FOR TREATING LYMPHOMA AND MYELOMA

(75) Inventors: Christine Dierks, Freiburg (DE); Markus Warmuth, Natick, MA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 12/439,878

(22) PCT Filed: Sep. 27, 2006
(86) PCT No.: PCT/US06/38171

§ 371 Date: Oct. 18, 2010

(51) Int. Cl. A61K 39/395; A61K 31/438; A61K 31/352; A61K 31/713; A61K 31/711; A61K 31/7105; C12N 5/09; A61P 35/00

(52) U.S. Cl. 424/143.1; 514/278; 514/455; 514/44 A; 514/44 R; 424/174.1; 435/375

(57) ABSTRACT

This invention relates to the use of antagonists of the hedgehog signaling pathway to induce apoptosis of lymphoma and myeloma cells and to treat subjects suffering from various forms of lymphoma or myeloma.

### FIELD OF THE INVENTION

The present invention generally relates to methods for inhibiting growth of tumor cells and for treating cancer.

### BACKGROUND OF THE INVENTION

Malignant lymphoma (ML) involves the cells of the lymphatic system, and is the fifth most common cancer in the U.S. ML includes Hodgkin's disease, and non-Hodgkin's diseases which are a heterogeneous group of lymphoid proliferative diseases. Hodgkin's disease accounts for approximately 14% of all malignant lymphomas. The non-Hodgkin's lymphomas are a diverse group of malignancies that are predominately of B-cell origin. In the Working Formulation classification scheme, these lymphomas been divided into low-, intermediate-, and high-grade categories by virtue of their natural histories (see “The Non-Hodgkin's Lymphoma Pathologic Classification Project,” Cancer 49:2112-2135, 1982). The low-grade lymphomas are indolent, with a median survival of 5 to 10 years (Homing and Rosenberg, N. Engl. J. Med. 311:1471-1475, 1984). Although chemotherapy can induce remissions in the majority of indolent lymphomas, cures are rare and most patients eventually relapse, requiring further therapy. The intermediate- and high-grade lymphomas are more aggressive tumors, but they have a greater chance for cure with chemotherapy. However, a significant proportion of these patients will relapse and require further treatment.

Multiple myeloma (MM) is malignant tumor composed of plasma cells of the type normally found in the bone marrow. These malignant plasma cells accumulate in bone marrow and typically produce monoclonal IgG or IgA molecules. The malignant plasma cells home to and expand in the bone marrow causing anemia and immunosuppression due to loss of normal hematopoiesis. Individuals suffering from multiple myeloma often experience anemia, osteolytic lesions, renal failure, hypercalcemia, and recurrent bacterial infections. MM represents the second most common hematopoietic malignancy.

There is an unmet need in the art for better treatment for lymphoma and myeloma. The present invention addresses this and other needs.

### SUMMARY OF THE INVENTION

In one aspect, the invention provides methods for inducing apoptosis of lymphoma or myeloma cells. These methods involve contacting the cells with an agent that inhibits hedgehog signaling pathway. Some of the methods are directed to inducing apoptosis of tumor cells that are present in a subject. Some of the methods are directed to inducing apoptosis of lymphoma or myeloma cells that do not express Gli3. Some of the methods employ an organic compound that specifically inhibits the hedgehog signaling pathway, e.g., cyclopamine or forskolin. Some other methods employ a nucleic acid agent (e.g., siRNA) that specifically inhibits expression of a hedgehog signaling pathway member, e.g., Smoothened, Suppressor of Fused, or transcription factor Gli (e.g., Gli1 or Gli2). Some of the methods employ an antagonist antibody that specifically binds to the transmembrane receptor Ptch.

In a related aspect, the invention provides methods for treating or ameliorating lymphoma or myeloma in a subject. The methods entail administering to the subject a pharmaceutical composition that contains an effective amount of an agent which down-regulates hedgehog signaling pathway. The agent can be an organic compound that specifically inhibits the hedgehog signaling pathway, e.g., cyclopamine or forskolin. The agent can also be a nucleic acid agent that specifically inhibits expression of a hedgehog signaling pathway member (Smoothened, Suppressor of Fused, or Gli). The agent can also be an antagonist antibody that specifically binds to the transmembrane receptor Ptch. In some of the methods, the subject is pre-screened for lack of Gli3 expression in the lymphoma or myeloma.

A further understanding of the nature and advantages of the present invention may be realized by reference to the remaining portions of the specification and claims.

### DESCRIPTION OF THE DRAWINGS

FIGS. 1A-1F show that hedgehog is a survival factor for lymphomas provided by bone marrow stroma cells. A: Alamar Blue Assay after 48 h of cultivation of lymphoma cells (mixed, B-cell lymphoma, plasmacytoma, plasmoblastoma) in medium containing RPMI 1640, FBS 10% and different cytokines in concentrations of 0, 1, 5, 10, 50 ng/ml shows increased number of viable cells in sonic hedgehog (Shh) and indian hedgehog (Ihh) stimulated cells; B: Growth of lymphoma cells with Shh 10 μM over 48 h can be inhibited by Hh pathway inhibition with anti-Hh 5E1 monoclonal antibody (10 μg/ml or Cyclopamine (5 μM) measured by fluorescence with an Alamar Blue assay; C: Expression of hedgehog protein in bone marrow stroma of different mouse strains (antibody recognizes Ihh and Shh); D: Transcription of Ihh in bone marrow of different mouse strains detected by rt-PCR; E: Similar proliferation rate of luciferased lymphoma cell lines on bone marrow stroma from different mouse strains (Balb/C, C57B16, B16 Cdkn2a−/−). Luminescence was measured after 0 h, 24 h and 48 h after seeding of 2×10E4 luciferased lymphoma cells on the stroma cells; and F: RT-PCR analysis of transcripts from Hh pathway members and targets in spleen lymphocytes and different lymphoma cell lines.

FIGS. 2A-2G show that Hh pathway inhibition induces apoptosis in stroma dependant lymphoma cells. A: Clearance of lymphoma cells from the stromal layer after 48 hours of cyclopamine treatment (5 μM); B: Treatment of luciferased lymphoma cells growing on an unluciferased stromal layer with cyclopamine, SANT-1 and tomatidine (0, 0.5, 1, 5 and 10 μM). Shown are luciferase readout with Bright glow luminescence reagent after 48 h treatment of lymphoma cells on stromal layer and detection of stroma growth alone by Alamar Blue assay after 48 hours treatment; C: Annexin V staining after treatment of lymphoma cells with 5 μM cyclopamine for 0 h, 12 h and 24 h; D: Cell cycle distribution of gated viable cells after treatment with cyclopamine for 0 h, 12 h and 24 h (SubG1 phase excluded from the picture); E: Expression of essential Hh pathway members (Smo, Ptc1, Ptc2, Gli1, Gli2) in B-cell lymphomas, plasmoblastomas and plasmacytomas; F: Down-regulation of Gli1 and Bcl2 protein in lymphoma cells after treatment with cyclopamine compared to actin control; and G: Quantitative RT-PCR for Ptch1 and BMI1 transcript levels after treatment of cells with 5 μM cyclopamine for 36 hours.

FIGS. 3A-3E show that overexpression of hedgehog pathway members and Bcl2 can inhibit cyclopamine induced apoptosis in Myc lymphomas. A: Overexpression of Gli1 and Fused in Myc-Ly6 rescues lymphoma cells from cyclopamine (5 μM) induced apoptosis shown after 48 h treatment; B: Luciferase assay with Myc-Ly6 cells overexpressing different Hh pathway members in 96 wells on stromal layer after 48 h treatment with cyclopamine in different concentrations. IC50 shift in cells overexpressing Smo, Smo535 and Smo562; reduced apoptosis induction in cells overexpressing Gli1 or Fused; C: Cyclopamine (5 μM) treatment of Myc⁺/p53^{-/-} lymphoma cells and Myc⁺/Cdkn2a^{-/-} lymphoma cells for 48 h induces apoptosis, while Myc⁺/Bcl2⁺ cells are resistant to cyclopamine treatment shown by fluorescence imaging of GFP positive cells. Partial resistance of Myc⁺/Bax^{-/-} cells and Myc⁺/Caspase3^{-/-} cells; D: Luciferase assay after 48 h treatment with cyclopamine; and E: Stroma withdrawal for 96 hours induces apoptosis in Myc/p53^{-/-} cells, Myc/Cdkn2a^{-/-} cells. Myc⁺/Bcl2⁺ cells grow stroma independent shown by light microscopy images.

FIGS. 4A-4F show that hedgehog pathway inhibition abrogates lymphoma expansion in vivo. A: Injection of 1 Mio luciferased Eμ-Myc lymphoma cells into C57B16 mice and start of treatment with cyclopamine or vehicle control day 2 after injection. Luciferase Xenogen imaging of mice after 12 days showed inhibition of lymphoma growth in cyclopamine treated mice; B: Survival curves for mice treated with vehicle control or cyclopamine for a maximum of 21 days; C: Survival curve for mice injected with Myc-Ly6 or Myc-Ly7 (expression of Gli3 and in vitro resistance to Hh inhibition) and treatment with Cyclopamine 50 mg/kg/d or vehicle control. D: injection of 1 Mio luciferased lymphoma cells in C57B16 mice. Xenogen imaging after a 3 day treatment with cyclopamine 50 mg/kg/biday in mice with fully developed lymphomas; E: Spleen weight and liver weight comparison after a 3 day treatment with cyclopamine; and F: H&E, Ki67 and PARP immunostaining of spleens isolated from vehicle and cyclopamine treated mice.

### DETAILED DESCRIPTION

### I. Overview

This invention is predicated in part on the discoveries by the present inventors that lymphoma and multiple myeloma diseases are dependent on the hedgehog (Hh) signaling pathway. As detailed in the Examples below, the inventors used lymphoma and plasmacytoma cells isolated from transgenic Eμ-Myc mice and Cdkn2a knockout mice, and discovered that hedgehog ligands mediate the interaction between stroma and lymphoma cells. The same was found for lymphoma and multiple myeloma samples isolated from patient samples from the bone (multiple myeloma) or from lymph nodes, bone marrow or spleens from non-Hodgkin's lymphoma (NHL) patients and also for chronic lymphocytic leukemia (CLL) samples. In addition, it was found that inhibition of the Hh signaling pathway induces apoptosis of stroma dependent lymphoma cells, and that overexpression of hedgehog pathway members inhibit cyclopamine induced apoptosis of lymphoma cells in vitro. Further, the inventors found that treating mice with hedgehog pathway inhibitors abrogates lymphoma expansion in vivo. Finally, the inventors discovered that there is no expression of Gli3 in spleen B-cells and in the majority of cyclopamine responsive lymphomas, but a predominant expression in all cyclopamine resistant lymphomas.

These data indicate that Hh signaling provides an important anti-apoptotic signal for the initial steps of transformation by c-Myc and plays an important role for lymphoma maintenance. Thus, disruption of the Hh signaling pathway provides novel means for treating lymphomas (e.g., NHL), multiple myelomas, CLL and other hematopoietic malignancies. In addition, expression of Gli3 in lymphomas provides a negative predictive factor for responsiveness to Hh inhibition and an important means for patient stratification.

In accordance with these discoveries, the invention provides methods for inhibiting growth of tumor cells, e.g., lymphoma and myeloma cells. The invention provides methods and compositions to treat lymphoma or myeloma in a subject by inhibiting growth of tumor cells. The methods are also useful to prevent tumorigenesis in a subject. Some of the methods are directed to treating lymphomas which do not have significant expression of Gli3 relative to spleen B cells. Typically, the methods involve administering to the subject in need of treatment a pharmaceutical composition that contains an antagonizing agent of Hh signaling (e.g., siRNAs, antibodies or small molecule organic compounds). These agents down-regulates cellular level or inhibits a biological activity of an Hh signaling pathway member.

The antagonists of Hh signaling can also be administered in combination with other therapies, such as radiation therapy, bone marrow transplantation, or hormone therapy. Subjects in need of treatment of lymphomas, myelomas or leukemia can be administered a hedgehog-antagonizing agent together with the administration of other therapeutic compounds to provide synergistic effects. These therapeutic compounds may be chemotherapeutic agents, ablation or other therapeutic hormones, antineoplastic agents, monoclonal antibodies useful against lymphomas or myelomas. Some of the well known anti-cancer drugs are described in the art, e.g., Cancer Therapeutics: Experimental and Clinical Agents, Teicher (Ed.), Humana Press (1^{st} ed., 1997); and Goodman and Gilman's The Pharmacological Basis of Therapeutics, Hardman et al. (Eds.), McGraw-Hill Professional (10^{th} ed., 2001). Examples of suitable anti-cancer drugs include 5-fluorouracil, vinblastine sulfate, estramustine phosphate, suramin and strontium-89. Examples of suitable chemotherapeutic agents include Asparaginase, Bleomycin Sulfate, Cisplatin, Cytarabine, Fludarabine Phosphate, Mitomycin and Streptozocin.

The following sections provide further guidance for practicing the methods of the invention, and for making and using the compositions of the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Oxford Dictionary of Biochemistry and Molecular Biology, Smith et al. (eds.), Oxford University Press (revised ed., 2000); Dictionary of Microbiology and Molecular Biology, Singleton et al. (Eds.), John Wiley & Sons (3^{rd} ed., 2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more molecules (e.g., a small molecule organic compound and a polypeptide) or combining molecules and cells (e.g., a compound and a cell). Contacting can occur in vitro, e.g., combining two or more agents or combining a compound and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

The term “hedgehog” is used to refer generically to any member of the hedgehog family, including sonic, indian, desert and tiggy winkle. The term may be used to indicate protein or gene. The term is also used to describe homolog/ortholog sequences in different animal species.

The terms “hedgehog (Hh) signaling pathway” and “hedgehog (Hh) signaling” are used interchangeably and refer to the chain of events normally mediated by various members of the signaling cascade such as hedgehog, patched (Ptch), smoothened (Smo), and Gli. The hedgehog pathway can be activated even in the absence of a hedgehog protein by activating a downstream component. For example, overexpression of Smo will activate the pathway in the absence of hedgehog.

Hh signaling components or members of Hh signaling pathway refer to gene products that participate in the Hh signaling pathway. An Hh signaling component frequently materially or substantially affects the transmission of the Hh signal in cells/tissues, typically resulting in changes in degree of downstream gene expression level and/or phenotypic changes. Hh signaling components, depending on their biological function and effects on the final outcome of the downstream gene activation/expression, may be divided into positive and negative regulators. A positive regulator is an Hh signaling component that positively affects the transmission of the Hh signal, i.e., stimulates downstream biological events when Hh is present. Examples include hedgehog, Smo, and Gli. A negative regulator is an Hh signaling component that negatively affects the transmission of the Hh signal, i.e., inhibits downstream biological events when Hh is present. Examples include (but are not limited to) Ptch and SuFu.

Hedgehog signaling antagonists, antagonists of Hh signaling or inhibitors of Hh signaling pathway refer to agents that inhibit the bioactivity of a positive Hh signaling component (such as hedgehog, Ptch, or Gli) or down-regulate the expression of the Hh signaling component. They also include agents which up-regulate a negative regulator of Hh signaling component. A hedgehog signaling antagonists may be directed to a protein encoded by any of the genes in the hedgehog pathway, including (but not limited to) sonic, indian or desert hedgehog, smoothened, ptch-1, ptch-2, gli-1, gli-2, gli-3, etc.

A “heterologous sequence” or a “heterologous nucleic acid,” as used herein, is one that originates from a source foreign to the particular host cell, or, if from the same source, is modified from its original form. Thus, a heterologous gene in a host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology.

A “host cell” refers to a prokaryotic or eukaryotic cell into which a heterologous polynucleotide can be introduced. The polynucleotide can be introduced into the cell by any means, e.g., electroporation, calcium phosphate precipitation, microinjection, transformation, viral infection, and/or the like.

The term “inhibiting” or “inhibition,” in the context of tumor growth or tumor cell growth, refers to delayed appearance of primary or secondary tumors, slowed development of primary or secondary tumors, decreased occurrence of primary or secondary tumors, slowed or decreased severity of secondary effects of disease, or arrested tumor growth and regression of tumors. The term “prevent” or “prevention” refers to a complete inhibition of development of primary or secondary tumors or any secondary effects of disease. In the context of modulation of enzymatic activities, inhibition relates to reversible suppression or reduction of an enzymatic activity including competitive, uncompetitive, and noncompetitive inhibition. This can be experimentally distinguished by the effects of the inhibitor on the reaction kinetics of the enzyme, which may be analyzed in terms of the basic Michaelis-Menten rate equation. Competitive inhibition occurs when the inhibitor can combine with the free enzyme in such a way that it competes with the normal substrate for binding at the active site. A competitive inhibitor reacts reversibly with the enzyme to form an enzyme-inhibitor complex [EI], analogous to the enzyme-substrate complex.

The term “sequence identity” in the context of two nucleic acid sequences or amino acid sequences refers to the residues in the two sequences which are the same when aligned for maximum correspondence over a specified comparison window. A “comparison window” refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). The CLUSTAL program is well described by Higgins and Sharp (1988) Gene 73:237-244; Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment. In one class of embodiments, the polypeptides herein are at least 70%, generally at least 75%, optionally at least 80%, 85%, 90%, 95% or 99% or more identical to a reference polypeptide, e.g., a hedgehog molecule described herein, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 95%, 99% or more identical to a reference nucleic acid, e.g., as measured by BLASTN (or CLUSTAL, or any other available alignment software) using default parameters.

A “substantially identical” nucleic acid or amino acid sequence refers to a nucleic acid or amino acid sequence which comprises a sequence that has at least 90% sequence identity to a reference sequence using the programs described above (preferably BLAST) using standard parameters. The sequence identity is preferably at least 95%, more preferably at least 98%, and most preferably at least 99%. For example, the BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915, 1989). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “modulate” with respect to a biological activity of a reference protein (e.g., a hedgehog pathway member) or its fragment refers to a change in the expression level or other biological activities of the protein. For example, modulation may cause an increase or a decrease in expression level of the reference protein, enzymatic modification (e.g., phosphorylation) of the protein, binding characteristics (e.g., binding to another molecule), or any other biological (e.g., enzymatic), functional, or immunological properties of the reference protein. The change in activity can arise from, for example, an increase or decrease in expression of one or more genes that encode the reference protein, the stability of an mRNA that encodes the protein, translation efficiency, or from a change in other biological activities of the reference protein. The change can also be due to the activity of another molecule that modulates the reference protein (e.g., a kinase which phosphorylates the reference protein).

Modulation of a reference protein can be up-regulation (i.e., activation or stimulation) or down-regulation (i.e. inhibition or suppression). The mode of action of a modulator of the reference protein can be direct, e.g., through binding to the protein or to genes encoding the protein, or indirect, e.g., through binding to and/or modifying (e.g., enzymatically) another molecule which otherwise modulates the reference protein.

The term “subject” includes mammals, especially humans. It also encompasses other non-human animals such as cows, horses, sheep, pigs, cats, dogs, mice, rats, rabbits, guinea pigs, monkeys.

The term “treat” or “treatment” refers to arrested tumor growth, and to partial or complete regression of tumors. The term “treating” includes the administration of compounds or agents to prevent or delay the onset of the symptoms, complications, or biochemical indicia of a disease (e.g., lymphoma and myeloma), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

A “variant” of a reference molecule refers to a molecule substantially similar in structure and biological activity to either the entire reference molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

### III. Antagonists of Hedgehog Signaling Pathway to be Employed

The therapeutic methods of the invention employ an antagonist of the hedgehog signaling pathway to inhibit growth and proliferation of lymphoma cells, leukemia cells, or myeloma cells. These methods involve contacting such a tumor cell (in vitro or in vivo) with an inhibitor of the Hh signaling pathway. Various types of inhibitors or antagonists of the hedgehog signaling pathway can be used to practice the methods. These include organic compounds that directly or indirectly modulate a biological activity (e.g., enzymatic activity) of a member of the hedgehog signaling pathway. They also include agents that specifically target a gene or a mRNA which encode a member of the hedgehog signaling pathway. Other antagonists of hedgehog signaling pathway that can be employed to practice the methods include antibodies or other binding agents which target a member of the hedgehog signaling pathway (e.g., a transmembrane receptor).

The Hh signaling pathway has been well characterized in the art (see, e.g., Nybakken and Perrimon, Curr. Opin. Genet. Dev. 12, 503-511, 2002; and Lum et al., Science 299: 2039-2045, 2003). Briefly, in the absence of hedgehog ligands, the transmembrane receptor, Patched (Ptch), binds to Smoothened (Smo) and blocks Smo's function. This inhibition is relieved in the presence of ligands, which allows Smo to initiate a signaling cascade that results in the release of transcription factors Glis from cytoplasmic proteins fused (Fu) and Suppressor of Fused (SuFu). In the inactive situation, SuFu prevents Glis from translocating to the nucleus. In the active situation, Fu inhibits SuFu and Glis are released. Gli proteins translocate into the nucleus and control target gene transcription.

To practice the therapeutic methods of the invention, a number of Hh signaling pathway components can be modulated. These include positive regulators of Hh signaling which can be antagonized and negative regulators of Hh signaling which can be agonized. Hedgehog (Hh) (including, e.g., Ihh, Shh, and Dhh), Smoothened (Smo), and Gli are examples of positive regulators, while Patched (Ptch) and Suppressor of Fused (Fu) are negative regulators. All Hh signaling pathway genes in various species can be easily cloned based on sequences readily available from public and proprietary databases, such as GenBank, EMBL, or FlyBase.

Many inhibitors of the hedgehog signaling pathway are known in the art and can be readily employed in the practice of the hedgehog signaling pathway. Some of Hh signaling antagonists are small molecule compounds which target a key member of Hh pathway such as Smo, e.g., cyclopamine, SANT1 and Cur61414 (Katoh et al., Cancer Biol Ther. 4:1050-4, 2005; and Williams et al., Proc Natl Acad Sci USA. 100:4616-21, 2003). For example, cyclopamine inhibits hedgehog signaling pathway by directly binding to Smo. Other antagonists of Hh signaling indirectly inhibit Hh pathway by acting on another molecule which in turn affects Hh signaling. For example, forskolin activates protein kinase A which in turn blocks Hh signaling downstream of Smo (See, e.g., Yao et al., Dev Biol. 246:356-65, 2002). Additional organic compound inhibitors of Hh signaling have been described in, e.g., US patent applications US20060063779 (Gunzner et al., 2006), US20050222087 (Beachy, 2005) and US 20010034337 (Dudek et al., 2001). Any of these Hh signaling antagonists can be employed to carry out the therapeutic methods of the present invention. Some of the compounds can be obtained commercially (e.g., cyclopamine or SANT-1). Others can be easily synthesized using methods routinely practiced in the art of organic chemistry.

In some embodiments, the employed antagonist of Hh signaling is a binding agent which specifically inhibits activation of the Hh signaling pathway. For example, when not bound by its ligand, the transmembrane receptor Ptch binds to Smo and blocks its function. Thus, a binding agent which can inhibit or block hedgehog binding to Ptch can be used to antagonize Hh signaling. Antagonist antibodies or antibody homologs as well as other molecules such as soluble forms of the natural binding proteins for hedgehog are useful. Preferably, monoclonal antibodies such an anti-hedgehog or anti-patched antibody homolog are used to practice the methods of the invention. These antibodies should be able to block hedgehog binding to Ptch but do not activate Hh signaling. In some methods, an antibody that specifically binds to a hedgehog polypeptide is used. Using neutralizing antibodies against hedgehog to inhibit Hh signaling is well known and routinely practiced in the art. See, e.g., Ahlgren et al., Curr Biol. 9:1304-14, 1999; Cobourne et al., J Dent Res. 80:1974-9, 2001; Hall et al., Dev Biol. 255:263-77, 2003; and Berman et al., Nature. 425:846-51, 2003. An example of such hedgehog neutralizing antibodies is monoclonal antibody clone 5E1. This antibody can be obtained from Developmental Studies Hybridoma Bank, University of Iowa. As demonstrated in the Examples below, such antibodies are able to inhibit hedgehog induced proliferation of lymphoma cells.

In some other embodiments, soluble forms of binding agents derived from Ptch can be used. These include soluble Ptch peptides, Ptch fusion proteins, or bifunctional Ptch/Ig fusion proteins. Some of these soluble agents contain a polypeptide fragment with a sequence identical or substantially identical to that of a Ptch fragment that harbors its ligand binding site. For example, a soluble form of Ptch or a fragment thereof which binds to hedgehog can be employed to compete with Ptch on cells for binding to hedgehog, thereby blocking activation of Hh signaling. In addition, soluble hedgehog mutants that bind Ptch but do not elicit hedgehog-dependent signaling can also be used in the practice of the invention.

Some therapeutic applications directed to human subjects employ antibody antagonists of Hh pathway that are preferably of human origin. These include human antibodies, humanized antibodies, chimeric antibodies, Fab, Fab′, F(ab′)2 or F(v) antibody fragments, as well as monomers or dimers of antibody heavy or light chains or mixtures thereof. A chimeric antibody is an antibody homolog in which all or part of the hinge and constant regions of an immunoglobulin light chain, heavy chain, or both, have been substituted with the corresponding regions from a human immunoglobulin light chain or heavy chain. A humanized antibody is an antibody homolog which, in addition to having human constant region sequences, also has some or all of its non-CDR amino acid residues in the variable regions being replaced with corresponding amino acids from a human immunoglobulin. Human antibodies are antibody homologs in which all of the amino acids of an immunoglobulin light and heavy chain are derived from a human source.

Antibody homologs include intact antibodies consisting of immunoglobulin light and heavy chains linked via disulfide bonds. It also encompasses a protein comprising one or more polypeptides selected from immunoglobulin light chains, immunoglobulin heavy chains and antigen-binding fragments thereof which are capable of binding to one or more antigens (i.e., hedgehog or patched). The component polypeptides of an antibody homolog composed of more than one polypeptide may optionally be disulfide-bound or otherwise covalently crosslinked. Antibody homologs also include portions of intact antibodies that retain antigen-binding specificity, for example, Fab fragments, Fab′ fragments, F(ab′)2 fragments, F(v) fragments, heavy chain monomers or dimers, light chain monomers or dimers, dimers consisting of one heavy and one light chain, and the like. Thus, antigen-binding fragments, as well as full-length dimeric or trimeric polypeptides derived from the above-described antibodies are also useful in the practice of the present invention.

Anti-hedgehog and anti-Patched antibody homologs can be produced using methods well known in the art, e.g., Monoclonal Antibodies—Production, Engineering And Clinical Applications, Ritter et al., Eds., Cambridge University Press, Cambridge, UK, 1995; and Harlow and Lane, Antibodies, A Laboratory Manual, Cold Spring Harbor Press, 3^{rd} ed., 2000. Human monoclonal antibody homologs against hedgehog or patched can be prepared using in vitro-primed human splenocytes, as described by Boerner et al., J. Immunol. 147:86-95, 1991. Alternatively, they may be prepared by methods described in, e.g., Persson et al., Proc. Nat. Acad. Sci. USA 88: 2432-2436, 1991; Huang and Stollar, J. Immunol. Methods 141: 227-236, 1991; U.S. patent application Ser. No. 10/778,726 (Publication No. 20050008625); and U.S. Pat. Nos. 5,798,230 and 5,789,650. Humanized recombinant antibody homolog having the capability of binding to a hedgehog or patched protein can be generated using methods described in, e.g., Riechmann et al., Nature 332: 323-327, 1988; Verhoeyen et al., Science 239: 1534-1536, 1988; Queen et al., Proc. Nat. Acad. Sci. USA 86:10029, 1989; and Orlandi et al., Proc. Natl. Acad. Sci. USA 86:3833, 1989.

Some therapeutic methods of the invention employ nucleic acid agents that antagonize the hedgehog signaling pathway. Typically, these agents down-regulate expression of one or more genes encoding positive Hh signaling components such as hedgehog, Smo or Gli. These include double-stranded RNAs such as short interfering RNA (siRNA) and short hairpin RNA (shRNAs), microRNA (miRNA), anti-sense nucleic acid, and complementary DNA (cDNA). Interference with the function and expression of endogenous genes by double-stranded RNAs has been shown in various organisms such as C. elegans as described, e.g., in Fire et al., Nature 391:806-811, 1998; drosophilia as described, e.g., in Kennerdell et al., Cell 95:1017-1026, 1998; and mouse embryos as described, e.g., in Wianni et al., Nat. Cell Biol. 2:70-75, 2000. Such double-stranded RNA can be synthesized by in vitro transcription of single-stranded RNA read from both directions of a template and in vitro annealing of sense and antisense RNA strands. Double-stranded RNA can also be synthesized from a cDNA vector construct in which a target gene is cloned in opposing orientations separated by an inverted repeat. Following cell transfection, the RNA is transcribed and the complementary strands reannealed. To antagonize Hh signaling in the present invention, double-stranded RNA targeting a positive regulator of Hh signaling pathway can be introduced into a cell (e.g., a lymphoma cell) by transfection of an appropriate construct.

In some embodiments, siRNAs antagonists of Hh signaling are employed in the practice of the invention. The siRNA antagonists can modulate hedgehog signaling at any point in the hedgehog signaling pathway. For example, they can regulate Hh signaling by antagonizing hedgehog itself, or any other positive Hh signaling components such as Smo or Gli. SiRNAs are typically around 19-30 nucleotides in length, and preferably 21-23 nucleotides in length. They are double stranded, and may include short overhangs at each end. SiRNAs can be chemically synthesized or recombinantly produced using methods known in the art. Recombinant production of siRNAs in general involves transcription of short hairpin RNAs (shRNAs) that are efficiently processed to form siRNAs within cells. See, e.g., Paddison et al. Proc Natl Acad Sci USA 99:1443-1448, 2002; Paddison et al. Genes & Dev. 16:948-958, 2002; Sui et al. Proc Natl Acad Sci USA, 8:5515-5520, 2002; Brummelkamp et al. Science, 296:550-553, 2002; Caplen et al., Proc Natl Acad Sci USA 98:9742-9747, 2001; and Elbashir et al., EMBO J. 20:6877-88, 2001.

In some embodiments, the nucleic acid antagonists of Hh signaling are double stranded hairpin RNA. The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; and Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

### IV. Diseases and Conditions to be Treated

This invention provides methods of prophylactic or therapeutic treatment of cancers of the blood and lymphatic systems, including lymphomas, leukemia, and myelomas. The methods employ an antagonist of hedgehog signaling pathway to inhibit growth and proliferation of lymphoma cells, leukemia cells, or myeloma cells. Lymphoma is malignant tumor of lymphoblasts derived from B lymphocytes. Myeloma is a malignant tumor composed of plasma cells of the type normally found in the bone marrow. Leukemia is an acute or chronic disease that involves the blood forming organs. NHLs are characterized by an abnormal increase in the number of leucocytes in the tissues of the body with or without a corresponding increase of those in the circulating blood and are classified according to the type of leucocyte most prominently involved.

By way of example, subjects suffering from or at risk of development of lymphoma (e.g., e.g., B-cell lymphoma, plasmoblastoma, plasmacytoma or CLL) can be treated with methods of the invention. Preferably, the subject is a human being. The methods entail administering to the subject a pharmaceutical composition containing an effective amount of an agent that inhibits the hedgehog signaling pathway. The subject can be one who is diagnosed with lymphoma, with or without metastasis, at any stage of the disease (e.g., stage I to IV, Ann Arbor Staging System). Lymphomas suitable for treatment with methods of the invention include but are not limited to Hodgkin's disease and non-Hodgkin's disease. Hodgkin's disease is a human malignant disorder of lymph tissue (lymphoma) that appears to originate in a particular lymph node and later spreads to the spleen, liver and bone marrow. It occurs mostly in individuals between the ages of 15 and 35. It is characterized by progressive, painless enlargement of the lymph nodes, spleen and general lymph tissue. Classic Hodgkin's disease is divided into four subtypes: (1) nodular sclerosis Hodgkin's disease (NSHD); (2) mixed cellularity Hodgkin's disease (MCHD); (3) lymphocyte depletion Hodgkin's disease (LDHD); and (4) lymphocyte-rich classic Hodgkin's disease (cLRHD).

In some preferred embodiments, the present methods are used to treat non-Hodgkin's Lymphoma (NHL). Non-Hodgkin's disease is also called lymphosarcoma and refers to a group of lymphomas which differ in important ways from Hodgkin's disease and are classified according to the microscopic appearance of the cancer cells. Non-Hodgkin's lymphoma includes but is not limited to (1) slow-growing lymphomas and lymphoid leukemia (e.g., chronic lymphocytic leukemia, small lymphocytic leukemia, lymphoplasmacytoid lymphoma, follicle center lymphoma, follicular small cleaved cell, follicular mixed cell, marginal zone B-cell lymphoma, hairy cell leukemia, plasmacytoma, myeloma, large granular lymphocyte leukemia, mycosis fungoides, szary syndrome); (2) moderately aggressive lymphomas and lymphoid leukemia (e.g., prolymphocytic leukemia, mantle cell lymphoma, follicle center lymphoma, follicular small cleaved cell, follicle center lymphoma, chronic lymphocytic leukemia/prolymphocytic leukemia, angiocentric lymphoma, angioimmunoblastic lymphoma); (3) aggressive lymphomas (e.g., large B-cell lymphoma, peripheral T-cell lymphomas, intestinal T-cell lymphoma, anaplastic large cell lymphoma); and (4) highly aggressive lymphomas and lymphoid leukemia (e.g., B-cell precursor B-lymphoblastic leukemia/lymphoma, Burkitt's lymphoma, high-grade B-cell lymphoma, Burkitt's-like T-cell precursor T-lymphoblastic leukemia/lymphoma). The methods of the present invention can be used for adult or childhood forms of lymphoma, as well as lymphomas at any stage, e.g., stage I, II, III, or IV. The methods described herein can also be employed to treat other forms of leukemia, e.g., acute lymphocytic leukemia (ALL).

Some of the therapeutic methods of the invention are particularly directed to treating lymphomas or myelomas which do not express Gli3. As disclosed in the Examples below, it was observed that, while Gli1 and Gli2 were expressed in all lymphomas, detectable Gli3 expression was present mainly in lymphomas which were resistant to Hh pathway inhibition by cyclopamine. There is no expression of Gli3 in normal spleen B-cells and in the majority of cyclopamine responsive lymphomas. Thus, prior to treatment with Hh antagonists, subjects with lymphomas can be first examined for expression of Gli3 in a lymphoma cell sample obtained from the subject. Gli3 expression level in the sample can be compared to Gli3 expression level in normal spleen B cells obtained from the subject. Gli3 expression levels in the lymphoma or myeloma samples and the control cells can be determined using methods well known in the art, e.g., as described in the Examples below. A likely responsiveness to treatment with Hh antagonists described herein is indicated by the lack of detectable Gli3 expression in the lymphoma or myeloma samples or an expression level that is not significantly higher (e.g., not more than 25%, 50%, or 100% higher) than Gli3 expression level in the normal B cell. Other than being an additional step of the therapeutic methods of the invention, the pre-screening for lack of Gli3 expression can be used independently as a method for patient stratification.

In addition to lymphomas, the methods and compositions described above are also suitable for the treatment of myelomas. Multiple myeloma is a fatal neoplasm characterized by an accumulation of a clone of plasma cells, frequently accompanied by the secretion of Ig chains. Bone marrow invasion by the tumor is associated with anemia, hypogammaglobinemia, and granulocytopenia with concomitant bacterial infections. An abnormal cytokine environment, principally raised IL-6 and IL-1β levels, often results in increased osteoclasis leading to bone pain, fractures, and hypercalcemia. Despite aggressive chemotherapy and transplantation, multiple myeloma is a universally fatal plasma proliferative disorder.

The therapeutic methods described herein can be used in combination with other cancer therapies. In the case of treating lymphomas, the subject to be treated may be one who is receiving concurrently other treatment modalities against the lymphoma. The subject can be a lymphoma patient who had undergone a regimen of treatment (e.g., chemotherapy and/or radiation) and whose cancer is regressing. The subject may be a lymphoma patient who had undergone a regimen of treatment (e.g., surgery) and who appears to be clinically free of the lymphoma. The hedgehog signaling antagonists described herein can be administered adjunctively with any of the treatment modalities, such as but not limited to chemotherapy, radiation, and/or surgery. For example, they can be used in combination with one or more chemotherapeutic or immunotherapeutic agents, such as vincristine, prednisone, doxorubicin, bleomycin, vinblastine, methotrexate, dexamethasone and leucovorin. They can also be used after other regimen(s) of treatment is concluded.

The present methods can be used to treat primary, relapsed, transformed, or refractory forms of cancer. Often, patients with relapsed cancers have undergone one or more treatments including chemotherapy, radiation therapy, bone marrow transplants, hormone therapy, surgery, and the like. Of the patients who respond to such treatments, they may exhibit stable disease, a partial response (i.e., the tumor or a cancer marker level diminishes by at least 50%), or a complete response (i.e., the tumor as well as markers become undetectable). In either of these scenarios, the cancer may subsequently reappear, signifying a relapse of the cancer.

The subject may be one who has not yet been diagnosed with lymphoma but are predisposed to or at high risk of developing lymphoma as a result of genetic factors and/or environmental factors. The subject may also be one who displays characteristics that are associated with a high risk of lymphoma, such as nodules detected by computer tomographic scanning or suspect cells in biopsy and/or body fluids.

Depending on the subject, the therapeutic and healthful benefits range from inhibiting or retarding the growth of the lymphoma and/or the spread of the lymphoma to other parts of the body (i.e., metastasis), palliating the symptoms of the cancer, improving the probability of survival of the subject with the cancer, prolonging the life expectancy of the subject, improving the quality of life of the subject, and/or reducing the probability of relapse after a successful course of treatment (e.g., surgery, chemotherapy or radiation). The symptoms associated with lymphoma include painless swelling in one or more of the lymph nodes of the neck, collarbone region, armpits, or groin, chest pain, coughing, fatigue, shortness of breath, fever, drenching night sweats, weight loss, fatigue, appetite loss, red patches on the skin, and severely itchy skin, often affecting the legs/feet.

The effect of the hedgehog signaling antagonists described herein on development and progression of lymphoma can be monitored by any methods known to one skilled in the art, including but not limited to measuring: a) changes in the size and morphology of the tumor using imaging techniques such as a computed tomographic (CT) scan or a sonogram; and b) changes in levels of biological markers of risk for lymphoma.

### V. Pharmaceutical Compositions and Administration

The hedgehog-antagonizing compounds of the present invention can be administered alone under sterile conditions to a subject in need of treatment. More preferably, they are administered as an active ingredient of a pharmaceutical composition. Pharmaceutical compositions of the present invention typically comprise an effective amount of at least one hedgehog-antagonizing agent described herein together with one or more acceptable carriers thereof. The compositions can also contain a second therapeutic agent noted above, e.g., a chemotherapeutic agent or other anti-cancer agent. Pharmaceutically carriers enhance or stabilize the composition, or to facilitate preparation of the composition. Pharmaceutically acceptable carriers are determined in part by the particular composition being administered (e.g., nucleic acid, protein, or other type of compounds), as well as by the particular method used to administer the composition. They should also be both pharmaceutically and physiologically acceptable in the sense of being compatible with the other ingredients and not injurious to the subject. They may take a wide variety of forms depending on the form of preparation desired for administration, e.g., oral, sublingual, rectal, nasal, or parenteral. For example, an antitumor compound can be complexed with carrier proteins such as ovalbumin or serum albumin prior to their administration in order to enhance stability or pharmacological properties.

There are a wide variety of suitable formulations of pharmaceutical compositions of the present invention (see, e.g., Remington: The Science and Practice of Pharmacy, Mack Publishing Co., 20^{th} ed., 2000). Without limitation, pharmaceutically acceptable carriers include syrup, water, isotonic saline solution, 5% dextrose in water or buffered sodium or ammonium acetate solution, oils, glycerin, alcohols, flavoring agents, preservatives, coloring agents starches, sugars, diluents, granulating agents, lubricants, and binders, among others. The carrier may also include a sustained release material such as glyceryl monostearate or glyceryl distearate, alone or with a wax.

The pharmaceutical compositions can be prepared in various forms, such as granules, tablets, pills, suppositories, capsules, suspensions, salves, lotions and the like. The concentration of therapeutically active compound in the formulation may vary from about 0.1-100% by weight. Therapeutic formulations are prepared by any methods well known in the art of pharmacy. See, e.g., Gilman et al., eds., Goodman and Gilman's: The Pharmacological Bases of Therapeutics, 8th ed., Pergamon Press, 1990; Remington: The Science and Practice of Pharmacy, Mack Publishing Co., 20^{th} ed., 2000; Avis et al., eds., Pharmaceutical Dosage Forms: Parenteral Medications, published by Marcel Dekker, Inc., N.Y., 1993; Lieberman et al., eds., Pharmaceutical Dosage Forms: Tablets, published by Marcel Dekker, Inc., N.Y., 1990; and Lieberman et al., eds., Pharmaceutical Dosage Forms: Disperse Systems, published by Marcel Dekker, Inc., N.Y., 1990.

The therapeutic formulations can be delivered by any effective means that can be used for treatment. Depending on the specific antitumor agent to be administered, the suitable means include oral, nasal, pulmonary administration, or parenteral (including subcutaneous, intramuscular, intravenous and intradermal) infusion into the bloodstream. For parenteral administration, antitumor agents of the present invention may be formulated in a variety of ways. Aqueous solutions of the modulators may be encapsulated in polymeric beads, liposomes, nanoparticles or other injectable depot formulations known to those of skill in the art. Additionally, the compounds of the present invention may also be administered encapsulated in liposomes. The compositions, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such a diacetylphosphate, stearylamine, or phosphatidic acid, and/or other materials of a hydrophobic nature.

The therapeutic formulations can conveniently be presented in unit dosage form and administered in a suitable therapeutic dose. A suitable therapeutic dose can be determined by any of the well known methods such as clinical studies on mammalian species to determine maximum tolerable dose and on normal human subjects to determine safe dosage. Except under certain circumstances when higher dosages may be required, the preferred dosage of an antitumor agent of the present invention usually lies within the range of from about 0.001 to about 1000 mg, more usually from about 0.01 to about 500 mg per day. The preferred dosage and mode of administration of an antitumor agent can vary for different subjects, depending upon factors that can be individually reviewed by the treating physician, such as the condition or conditions to be treated, the choice of composition to be administered, including the particular antitumor agent, the age, weight, and response of the individual subject, the severity of the subject's symptoms, and the chosen route of administration. As a general rule, the quantity of an antitumor agent administered is the smallest dosage which effectively and reliably prevents or minimizes the conditions of the subjects. Therefore, the above dosage ranges are intended to provide general guidance and support for the teachings herein, but are not intended to limit the scope of the invention.

### EXAMPLES

The following examples are provided to illustrate, but not to limit the present invention.

### Example 1

### General Materials and Methods

Genetically altered mice and culture of primary cells: Eμ-Myc mice (Adams et al., Nature 318: 533-53824, 1985), Cdkna^{-/-} mice (Serrano et al., Cell. 85:27-3725, 1996), Bax^{-/-} mice (The Jackson Laboratory, Bar Harbor, Me.), Caspase3^{-/-} mice (The Jackson laboratory), p53^{-/-} mice (The Jackson Laboratory) and Bcl2 tg mice (The Jackson laboratory) were maintained and genotyped as described. Eμ-Myc mice and Cdkn2a−/− mice were monitored for signs of disease including development of visible lymphomas or weight loss from more than 15%. Mice with terminal disease were sacrificed, bone marrow, spleen and lymph nodes were extracted and lymphoma cells were propagated under Witlock/Witte culture conditions. For maintained growth cells were transferred on bone marrow stroma from Cdkn2a−/− mice after 2-3 weeks of culture. For generation of lymphomas with defined genetic background, bone marrow from p53−/− mice, Caspase3−/− mice, Bax−/− mice, Cdkn2a−/− mice and Bcl2 tg mice was extracted and pMSCV c-Myc IRES GFP was overexpressed. Propagation of transformed lymphocytes was performed under Witlick/Witte culture conditions and lymphomas were maintained on stroma from Cdkn2a−/− mice.

Cell culture experiments: Lymphoma cells were infected with a retrovirus containing a pMSCV IRES puro-luc sequence and selected for 7 days with puromycin on puromycin-resistant Cdkn2a−/− stroma. Cyclopamine was obtained from Toronto Research Chemicals and SANT-1 was obtained from EMDBioscience. Both were dissolved as x 1,000 stocks in DMSO. SCF was obtained from RDI, and all other cytokines were obtained from R&D systems. 5E1 anti-ShhN monoclonal antibody was obtained from the developmental hybridoma bank and was used at a concentration of 10 μg/ml. For cytokine stimulation and Shh inhibition in supernatant lymphoma cells were seeded into 96-well plates at a density of 20,000 cells per well and mitochondrial activity of viable cells was measured with an Alamar Blue Assay as described.

For Shh pathway inhibition on stroma, 6200 stroma cells were seeded per 96-well. After 24 hours 20,000 luciferased lymphoma cells were seeded on the stroma cells and 2 hours later compound or DMSO was added. Luciferase assay was performed with Bright-glow reagent as described. To generate cells overexpressing Hh pathway members, lymphoma cells were infected with Smo, Smo 535, Smo 562, Gli1 and Fused-pMSCV IRES GFP constructs and then sorted for GFP positive cells.

Immunohistochemistry: Single colour DAB-immunoperoxidase staining was performed as described. Antibodies were obtained from Santa Cruz Biotechnologies (for Shh antibody, N-19), NeoMarkers (for Ki67 antibody) and Promega (for PARP antibody). All staining was performed on paraffin sections from C57B16 mice after 3 day treatment with cyclopamine or vehicle control.

Western immunoblot: Whole-cell lysates were sonicated in 2% SDS/50 mM TrisHCl, pH8. Western blot using rabbit or goat polyclonal antibodies for Shh-N, c-Myc, Smo, Gli1 were performed as described. Shh-N, c-Myc and Smo antibodies were obtained from Santa Cruz Biotechnology, Gli1 (ab7523) antibody was obtained from Abcam Inc. (Cambridge, Mass.).

RT-PCR: Total cellular RNA was treated with DNase, reverse transcribed, amplified for 33 cycles at an annealing temperature of 55° C. Primers used were obtained from Integrated DNA Technologies (Coralville, Iowa).

Mouse experiments: Black6 mice between 6 and 8 weeks of age were injected with 1 Mio luciferased lymphoma cells. Treatment with Cyclopamine 25 mg/kg/d, 50 mg/kg/d, 100 mg/kg/d or vehicle control started day 2 or when lymphomas were already developed. Bioluminescence was measured with injection of Renilla luciferase.

### Example 2

### Hh Signaling is Required for Survival and Growth of Lymphoma Cells In Vitro

We investigated whether the hedgehog pathway is involved in malignant haematopoiesis, especially in the development and maintenance of malignant lymphoma. We used lymphoma cells isolated from transgenic mice overexpressing the c-Myc oncogene (Eμ-Myc mice; Adams et al., Nature 318: 533-538, 1985), as well as lymphoma cells isolated from Cdkn2a^{-/-} mice (loss of tumour suppressors p16INK4a and p19ARF; Serrano et al., Cell 85:27-37, 1996). These cells were employed as a genetically engineered model for lymphomagenesis. Bone marrow, lymph nodes and spleens were extracted from mice with clinical signs of disease such as enlarged lymph nodes or weight loss of more than 15% of their initial body weight. Lymphoma development occurred between 8 and 20 weeks in Eμ-myc mice and at age 15 to 30 weeks in Cdkn2a^{-/-} mice, respectively. Lymphoma cells were propagated on bone marrow stroma isolated from diseased mice for 3 weeks and then transferred to bone marrow stroma isolated from Cdkn2a^{-/-} mice for maintained growth in culture.

A total of 34 Myc-positive (Myc-lymphomas) and 8 Cdkn2a^{-/-} primary lymphoma cell cultures were established. Characterization of Eμ-Myc lymphomas by flow cytometry (B220, CD19, CD138), BCL6 immunohistochemistry and H&E staining showed 29.4% B-cell lymphomas (10/34), 35.2% plasmoblastomas (12/34), 29.4% plasmacytomas (10/34) and 5.8% mixed lymphomas (2/34). Cdkn2a^{-/-} lymphomas were characterized as 50% (4/8) plasmoblastomas and 50% (4/8) plasmacytomas. These results suggest that the same genetic lesion can give rise to multiple different lymphoma phenotypes that closely resemble human lymphomas derived from various stages of lymphocyte development, supposedly dependent on secondary mutations acquired during disease induction. The in vitro proliferation and survival of these lymphomas was dependent on the presence of a stromal layer, as shown by rapid induction of apoptosis within 24-36 h of cells taken off stroma. Interestingly, growth of lymphoma cells in the absence of a stromal layer could be sustained for at least 2-3 days by addition of supernatant produced by stroma cells, suggesting that a soluble factor secreted by stroma cells contributes to the growth and survival of these cells.

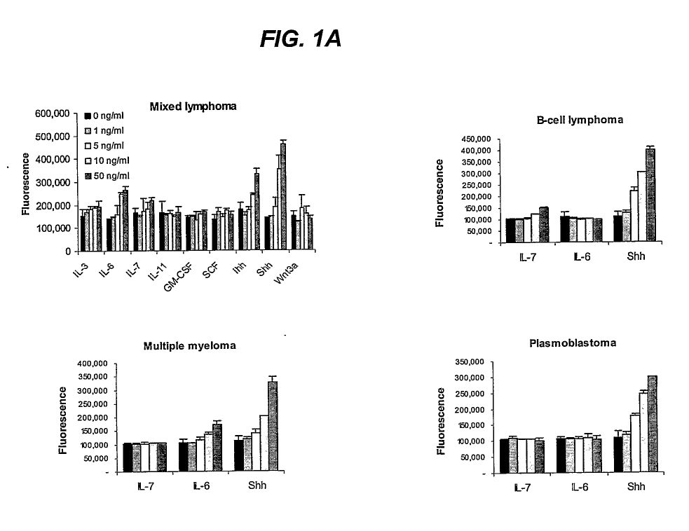

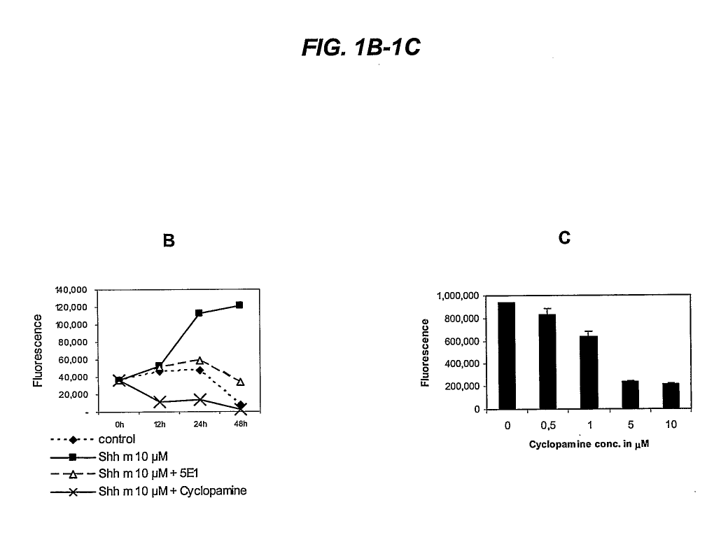

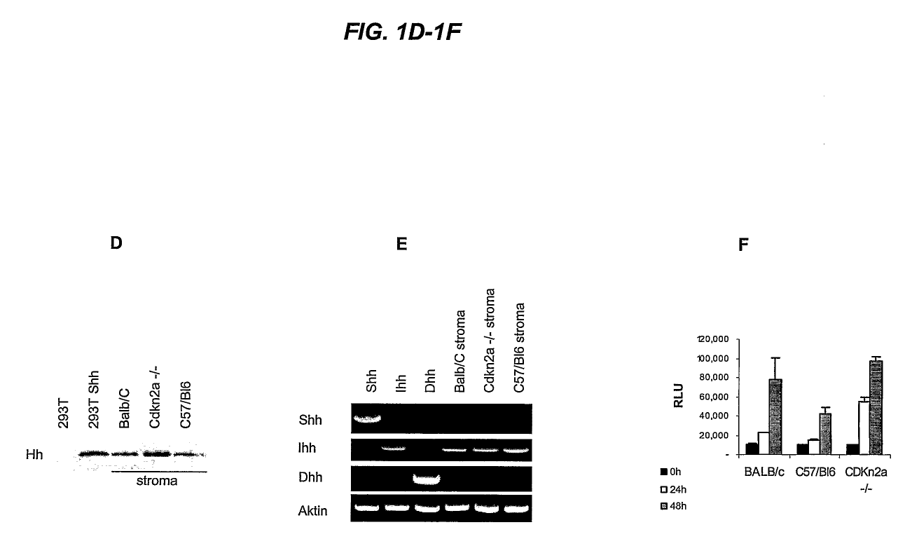

To identify growth stimuli secreted by stroma cells which are sufficient to maintain survival and growth of lymphoma cells in the absence of a stromal layer, lymphoma cells of various differentiation stages were grown in the presence of multiple different growth factors. Stimulation with either IL-6 or IL-7 and concurrent removal of the stroma cells could enhance survival of either plasmacytomas (IL-6) or B-cell lymphomas (IL-7) and mixed lymphomas (IL-6 and IL-7) but not plasmoblastomas (early plasma cells) (FIG. 1A). While no effect on lymphocyte growth was seen after stimulation with IL-3, IL-11, GM-CSF, SCF or Wnt3a, stimulation with recombinant Shh or Ihh could increase the number of viable lymphoma cells 2 days after stroma removal even more than IL-6 and IL-7 (FIG. 1A). Shh stimulation induced proliferation of Eμ-Myc lymphoma cells in the absence of a stromal layer for over 2 days (FIG. 1B). This effect could be inhibited by either disruption of Hh binding to its receptor PTC using the Hh-specific neutralizing antibody 5E1 or by abrogation of Hh signalling with cyclopamine, an alkaloid which specifically binds to SMO and stabilizes its inactive conformation (FIG. 1B). Hedgehog pathway inhibition also inhibited proliferation of lymphoma cells cultivated off stroma in the presence of supernatant generated from stroma cells (FIG. 1C). These results indicate that Hh family members might be important growth factors secreted by stroma cells to sustain the proliferation and survival of malignant lymphoma cells.

Analysis of bone marrow stroma from 3 different mouse strains (BALB/c, C57B16, Cdkn2a^{-/-}) showed expression of Hh protein. (FIG. 1D). Further analysis by RTPCR displayed expression of Ihh RNA, while no mRNA for Shh and Dhh could be detected in the stroma cells (FIG. 1C). In line with these results, lymphoma cells independent of their differentiation and origin (Eμ-Myc or Cdkn2a^{-/-}) could be cultured on all 3 different stroma layers and similar proliferation rates were observed (FIG. 1F).

### Example 3

### Inhibition of Hh Pathway Induces Apoptosis of Lymphoma Cells In Vitro

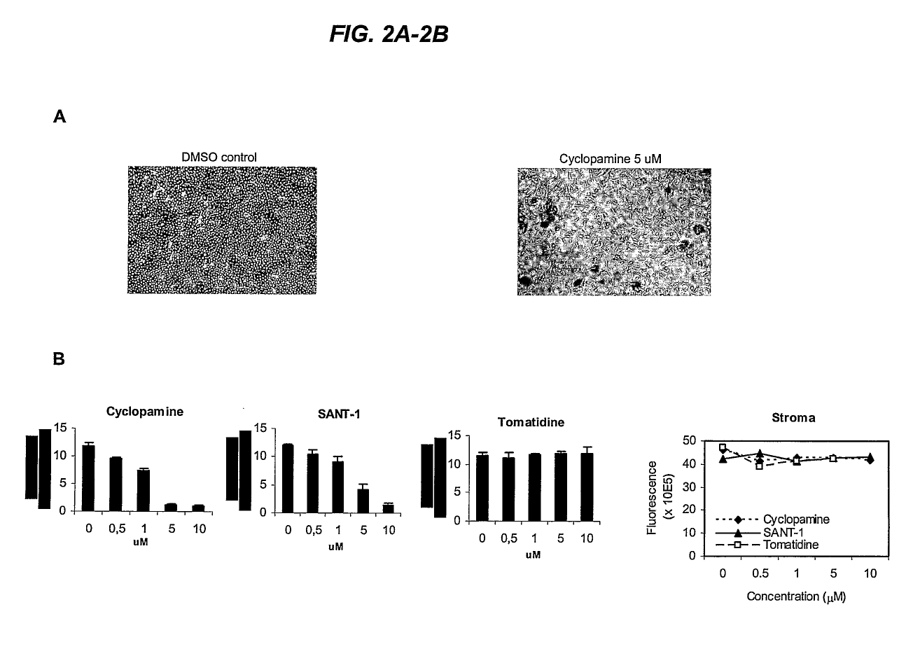

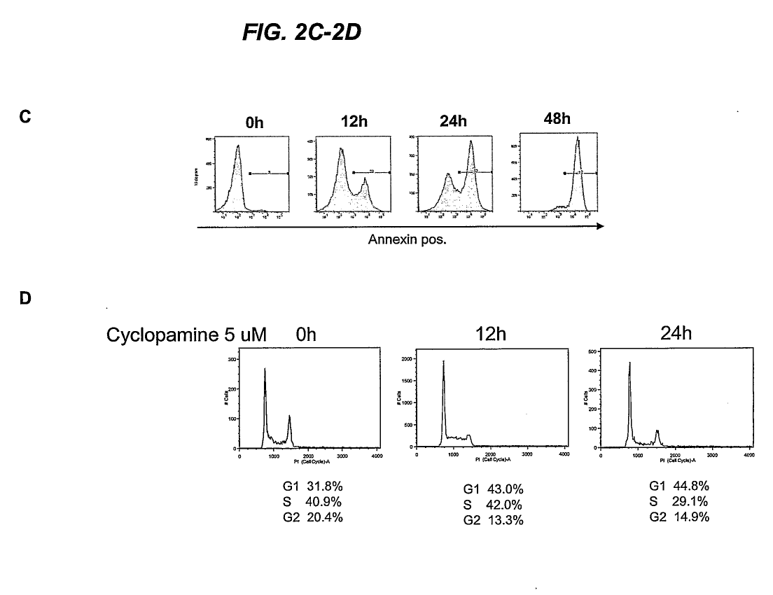

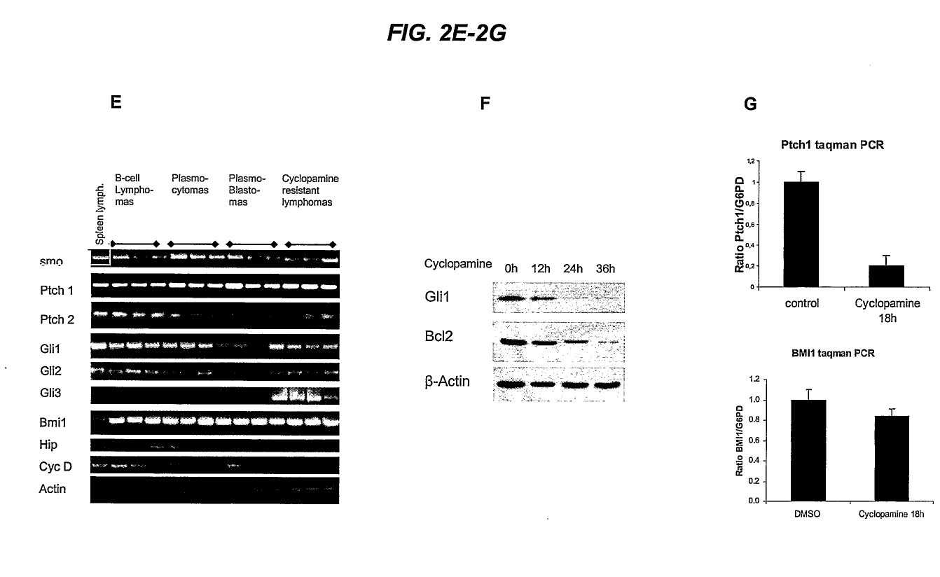

In order to investigate whether the Hh signalling pathway does indeed contribute to the survival and proliferation of lymphoma cells growing on an intact stromal layer, lymphoma cells were luciferased using a retrovirus expressing a puromycin/Luc fusion cassette. 2×10E4 lymphoma cells constitutively expressing luciferase were added to each 96-well containing a stromal layer (not expressing luciferase) isolated from Cdkn2a^{-/-} mice. A total of 34 individual Eμ-Myc-lymphomas and 8 Cdkn2a-lymphomas were tested. A luciferase assay was performed as a readout for cell viability and proliferation and results were also documented by light microscopy. As shown in FIG. 2A, treatment of a representative Myc-lymphoma (Myc-Ly4) with cyclopamine at a concentration of 5 μM resulted in a complete clearance of the lymphoma cells from the stromal layer within 48 h (FIG. 2A). This result indicate that Hh family members secreted by bone marrow stroma cells and subsequent activation of SMO and the Hh signalling cascade in these cells might contribute to the in-vitro expansion of the cells. As a control, it was observed that growth and survival of the stromal layer was not affected by cyclopamine treatment (FIG. 2A).

In addition, the viability of lymphoma cells (Myc-Ly6) was determined 48 hours after treatment with either cyclopamine, the Smo antagonist SANT-1 or tomatidine, an alkaloid with similar structure to cyclopamine, but no smoothened binding activity. Luciferase activity, representing viable lymphoma cells, was reduced in a dose dependant manner in both cyclopamine and SANT-1 treated cells, but not in tomatidine treated cells (FIG. 2B). The concentration needed for 50% of maximal inhibition (IC50) for cyclopamine was between 0.5 and 2 μM in most responsive lymphomas and 1-3 μM for SANT-1. Stroma growth was not inhibited by either of these compounds as demonstrated in an independent experiment using Alamar Blue as a readout (FIG. 2B).

Flow cytometry analysis for annexin V staining as a measurement for induction of apoptosis showed that Hh pathway inhibition in lymphoma cells by 5 μM cyclopamine induced a dramatic increase in apoptosis within 12 hours to more than 50% and nearly 100% apoptotic cells after 48 hours of treatment (FIG. 2C). In addition to apoptosis induction we could also detect a change in cell cycle distribution of the remaining viable cells, specifically a reduction of the G2/M phase from 20.4% to 13.3% after 12 hours of treatment (FIG. 2D). To further corroborate these findings, most lymphomas isolated from both Eμ-Myc and CDKN2a^{-/-} mice were tested for cyclopamine responsiveness. Table 1 summarizes the response rates of all tested lymphoma cultures. Cyclopamine sensitivity was defined as greater than 80% growth inhibition at a concentration of 5 μM. A total of 74.1% of Eμ-Myc-lymphomas and 50% of lymphomas isolated from CDKN2a^{-/-} mice were sensitive to Hh pathway inhibition. B-cell lymphomas and plasmoblastomas from Eμ-Myc mice had the highest response rates, with 80% (8/10) each, followed by plasmacytomas 66% (6/9) and mixed lymphomas 50% (1/2) (Table 1).

TABLE 1
Response rates of lymphoma cultures to cyclopamine treatment

| | | | | |
|---|---|---|---|---|
| Lymphoma | B-cell | Plasmo- | Plasma- | Mixed |
| type | lymphoma | blastoma | cytoma | lymphomas |

Myc-lymphomas: Phenotype and cyclopamine response

| | | | | |
|---|---|---|---|---|
| Percentage of | 29.4% | 35.2% | 29.4% | 5.8% |
| total | (10/34) | (12/34) | (10/34) | (2/34) |
| lymphomas | | | | |
| Response rate | 80% | 80% | 66% | 50% |
| to cyclopamine | (8/10) | (8/10) | 6/9 | 1/2 |
| treatment | | | | |

Ink4a/Arf-/- -lymphomas: Phenotype and cyclopamine response

| | | | | |
|---|---|---|---|---|
| Percentage of | 0% | 50% | 50% | 0% |
| total | | (4/8) | (4/8) | |
| lymphomas | | | | |
| Response rate | | 50% | 50% | |
| to cyclopamine | | (2/4) | (2/4) | |
| treatment | | | | |

To further substantiate a role for Hh/SMO signalling in Eμ-Myc and CDKN2a^{-/-} positive lymphoma we determined the expression of Hh pathway members in the lymphoma cells itself. Transcripts of all essential Hh pathway members (Smo, Ptc1, Ptc2, Gli1, and Gli2) were expressed in B-cell lymphomas, plasmoblastomas and plasmacytomas (FIG. 2E). Gli1 and Ptch represent direct target genes of the Hh pathway itself and are regulated at the transcriptional level by Hh signalling. Therefore, high expression of Gli1 and Ptch observed in most primary lymphoma cell cultures were suggestive of Hh pathway activation in these lymphoma cells. Inhibition of Hh signalling by cyclopamine in the lymphoma cells resulted in a reduction of detectable Gli1 protein in these cells within 24 hours compared to the actin control (FIG. 2F). Ptch1 transcript levels were decreased 10-fold within 18 h of pathway inhibition (FIG. 2G). Other target genes of the Hh pathway like Hip and Cyclin D1 could also be detected in the majority of lymphoma cells. Bcl2, another known downstream target of the pathway in T-lymphocytes, was downregulated in Myc-lymphomas after Cyclopamine treatment (FIG. 2F). Interestingly, while Gli1 and Gli2 were present in all lymphomas, Gli3 was mainly expressed in lymphomas which were resistant to Hh pathway inhibition by cyclopamine (FIG. 2E). All 3 Gli proteins have evolved specialized functions with distinct temporal and tissue-specific expression patterns. Unlike Gli1, which represents a direct transcriptional Hh-target gene, Gli2 and also Gli3 are considered latent transcriptional regulators activated by Hh signalling through modification of their N-terminal repressor domain.

Molecular analysis suggests that Gli3 (and Gli2) can be proteolytically processed into a repressor form (Gli3^{Rep}) that suppresses the Gli1 promoter, whereas the full-length form of Gli3 (FL-Gli3) directly mediates the activation of a Gli1 promoter in response to a Shh signal. Hedgehog pathway activation inhibits cleavage of Gli3 into its repressor form, but can also downregulate Gli3 expression dependent on tissue type and context. The regulation of Gli3 expression and its function is not fully understood and there is no data so far on its role in B-lymphocytes. Our results demonstrating that there is no expression of Gli3 in spleen B-cells and in the majority of cyclopamine responsive lymphomas, but a predominant expression in all cyclopamine resistant lymphomas, indicates aberrant pathway activation in this subclass of lymphomas. Finally, Bmi-1, a member of the Polycomb group of transcriptional repressors, which was shown to be essential for lymphoma development in transgenic Myc mice, was upregulated in all lymphomas compared to B-cells extracted from the spleen independent of the lymphoma type or responsiveness to hedgehog pathway inhibition.

### Example 4

### Up-Regulating Hh Pathway Inhibit Apoptosis in Myc Lymphoma Cell Lines

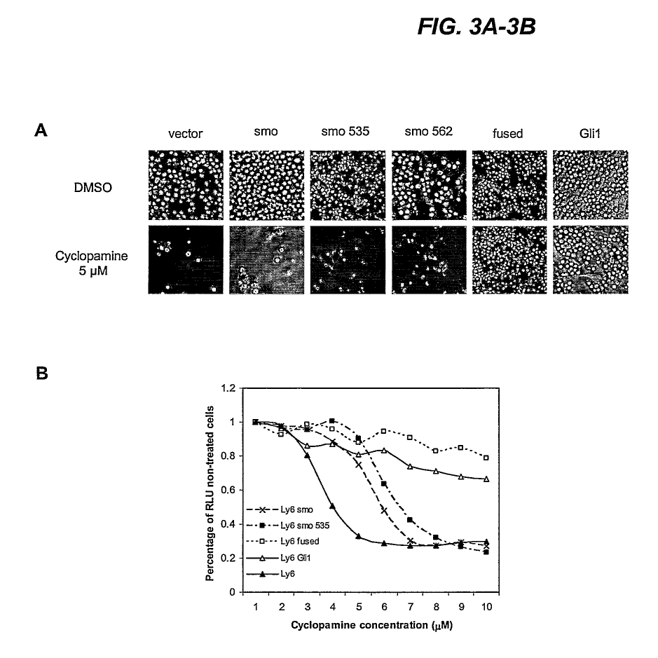

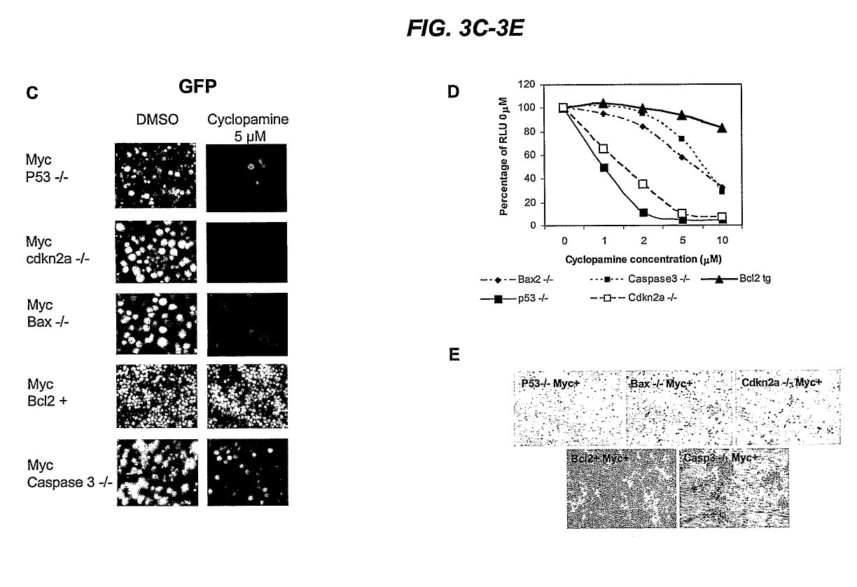

We examined whether the observed effect of cyclopamine on the growth and survival of lymphoma cells was mediated by specific Hh pathway inhibition or rather reflects off-target effects. Several different pathway members were overexpressed in two of the cyclopamine sensitive lymphoma cell lines (1 B-cell lymphoma and 1 plasmoblastoma) in an attempt to rescue cyclopamine induced inhibition of the pathway and inhibition of growth and survival. Overexpression of Smo wt and two constitutively active Smo mutants, Smo W535L and Smo A562G³⁷ in both lymphomas, led to a slight increase of the IC50 values for cyclopamine from 1 to 3, 3.5 and 4.5 μM respectively (FIGS. 3A and 3B). Similar effects were seen by concurrent stimulation of cells with a Smo agonist, purmorphamine, which is known to compete with cyclopamine for Smo binding (Sinha et al., Nat Chem Biol. 2:29-30, 2006). Overexpression of the downstream effector Fused lead to a nearly complete inhibition of apoptosis induction by cyclopamine while Gli1 overexpression lead to a partial resistance of Myc-Ly6 to cyclopamine treatment (FIGS. 3A and 3B) with the IC50 in both cases shifted beyond 10 μM. Similar effects were seen for both lymphomas in several independent experiments. These data indicate that the effect of cyclopamine on the growth and survival of lymphoma cells is directly linked to hedgehog pathway inhibition.

The hedgehog pathway was shown to play an important role in the regulation of both cell cycle and apoptosis of various types of cells. In addition, several lines of evidence suggest that Hh signalling regulates the expression of BMI-1, which in turn, by repressing the Ink4a/Arf locus influences processes as important as stem cell self renewal as well as proliferative and oncogenic senescence. To better understand which specific pathways are involved in the regulation of growth and survival through Hh signalling in lymphoma cells, lymphoma cell cultures were generated in multiple different genetic backgrounds in order to modify either apoptotic or failsafe pathways. Bone marrow extracted from wt C57BL/6, Bcl2 transgenic, Bax^{-/-} Caspase-3^{-/-}, CDKN2a^{-/-} and p53^{-/-} mice was infected with pMSCV-Myc-GFP and transformed lymphocyte cultures were obtained using Whitlock/Witte culture conditions in the absence of IL-7. All genetic backgrounds tested except bone marrow from normal C57BL/6 mice gave rise to transformed lymphocyte cell cultures growing in the absence of IL-7. All lymphocyte cell cultures established showed bright GFP fluorescence, and enhanced Myc levels could be detected by Western blotting. Similar to cultures established from primary lymphomas, lymphocytes transformed by retroviral transduction were transferred to bone marrow stroma of CDKN2a^{-/-} mice for longer term culture.

Treatment of Myc/p53^{-/-} lymphoma cells and Myc/Cdkn2a^{-/-} lymphoma cells with cyclopamine induced apoptosis within 48 hours (FIGS. 3C and 3D). In marked contrast, no reduction of viable cells could be detected in Myc/Bcl2⁺ lymphoma cells. Myc/Bax−/− cells and Myc/Caspase 3^{-/-} cells showed a partial resistance to cyclopamine induced apoptosis (FIG. 3D). Stroma removal resulted in apoptosis induction in Myc/p53^{-/-} lymphoma cells and Myc/Cdkn2a^{-/-} lymphoma cells, while Myc/Bcl2⁺ lymphoma cells were not affected (FIG. 3E). These results suggest that the apoptotic cell death seen on lymphocytes after stroma removal or on treatment with cyclopamine is mediated through the classical mitochondrial apoptosis pathway and does not involve regulation of p53 through the Ink4/Arf locus or Bmi1.

### Example 5

### Inhibiting Hh Pathway Abrogates Lymphoma Expansion In Vivo

The data described above indicate the importance of Hh signalling for the growth and survival of primary lymphoma cells under in-vitro culture conditions. Lymphoma growth in vivo is primarily restricted to lymphatic organs such as the spleen, lymph nodes and bone marrow. The stroma of all these organs expresses either Shh or Ihh and therefore the growth and expansion of lymphomas in-vivo is likely to be regulated by Hh signalling as well.

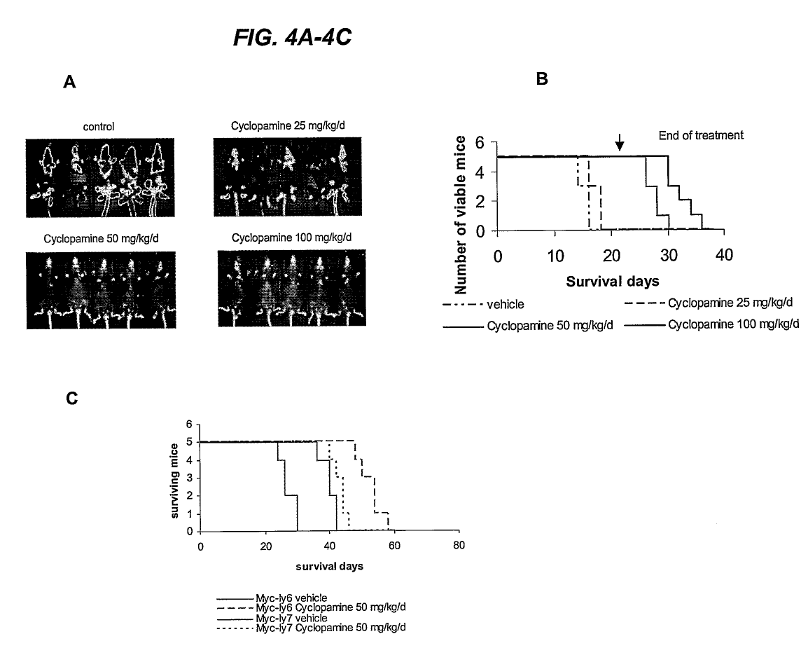

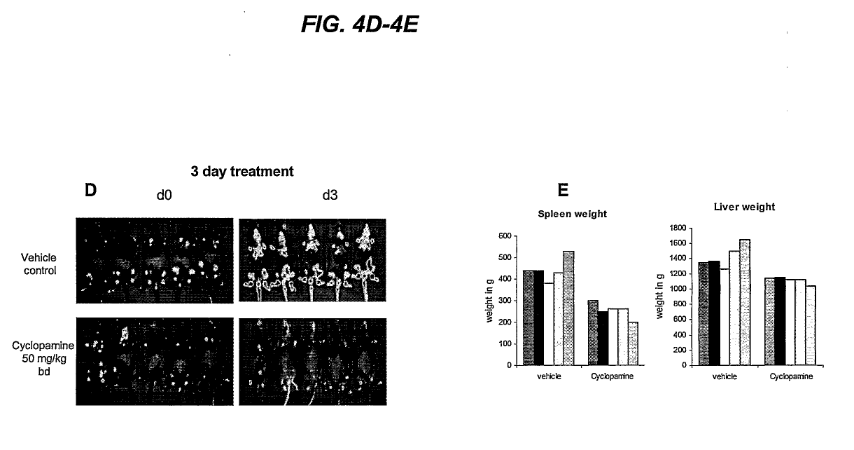

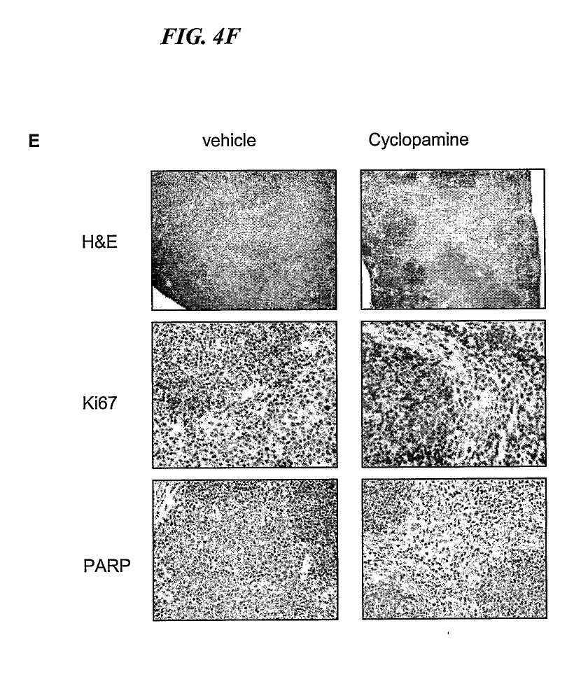

In order to verify the importance of the Hh pathway for lymphoma growth and expansion in mice, we injected 1e6 lymphoma cells expressing luciferase into syngeneic C57BL/6 mice. On day 2 post-injection, mice were treated with either vehicle control or cyclopamine (100, 50 or 25 mg/kg/day) for a maximum of 21 days by subcutaneous injection. Luciferase levels were measured by bioluminescence imaging twice a week. Twelve days post-injection, the control group showed high luminescence in the lymph nodes and spleen of all injected mice (FIG. 4A). Mice treated with cyclopamine doses of 50 and 100 mg/kg/day had only minimal signs of disease (FIG. 4A). Median survival of mice in the control group was 16 days, while survival of cyclopamine treated mice was enhanced to 20 days in the lowest dosing group (25 mg/kg/day) and 30 to 35 days with cyclopamine doses of 50 and 100 mg/kg/day (FIG. 4B). Two additional lymphoma cells (1 plasmoblastoma and 1 B-cell lymphoma) were shown to be responsive in vivo under similar conditions, while one lymphoma with endogenous resistance to cyclopamine treatment in vitro and associated overexpression of Gli3, showed only minimal response to cyclopamine treatment at 100 mg/kg/day shown by survival curves (FIG. 4C).

We next wished to determine the importance of Hh signalling in established lymphomas. Therefore, in a separate experiment, cyclopamine treatment was started 10 days after injection of lymphoma cells (Ly9) at a time when visible signal could be detected from all mice. Cyclopamine treatment with 50 mg/kg twice a day for 3 days significantly reduced light emission from mice, markedly decreased the infiltration of the spleen, and reduced lymphoma mass in lymph nodes and other organs compared to the control group (FIG. 4D). Spleen weight following a three-day cyclopamine treatment was decreased by nearly 50% compared to the vehicle control group (FIG. 4E), and the liver weight in the treated mice was reduced to an average weight from 900 to 1000 g (FIG. 4E). Histopathological analysis of spleens isolated from vehicle and cyclopamine treated mice showed a reduction of lymphoma cells in the spleen after 3 days of treatment with cyclopamine (FIG. 4F). Ki67 staining showed a strong decrease of high proliferative cells in the spleen compared to the vehicle group, and PARP staining showed apoptosis induction in the areas around the white pulpa (FIG. 4F).

It is understood that the examples and embodiments described herein are for illustrative purposes only and that various modifications or changes in light thereof will be suggested to persons skilled in the art and are to be included within the spirit and purview of this application and scope of the appended claims.

All publications, patents, patent applications, polynucleotide and polypeptide sequence accession numbers and other documents cited herein are hereby incorporated by reference in their entirety and for all purposes to the same extent as if each of these documents were individually so denoted.

We claim:

1. A method for inducing apoptosis of a lymphoma or myeloma cell, the method comprising contacting the cell with an agent that inhibits hedgehog signaling pathway.

2. The method of claim 1, wherein the cell is present in a subject.

3. The method of claim 1, wherein the cell does not express Gli3.

4. The method of claim 1, wherein the agent is an organic compound that specifically inhibits the hedgehog signaling pathway.

5. The method of claim 4, wherein the compound is cyclopamine or forskolin.

6. The method of claim 1, wherein the agent is a nucleic acid agent that specifically antagonizes a hedgehog signaling pathway member.

7. The method of claim 6, wherein the hedgehog signaling pathway member is Smoothened (Smo), Suppressor of Fused (SuFu), or transcription factor Gli.

8. The method of claim 6, wherein the agent is selected from the group consisting of an short interfering RNA (siRNA), a short hairpin RNA (shRNA), a microRNA (miRNA), an anti-sense nucleic acid, and a complementary DNA (cDNA).

9. The method of claim 8, wherein the siRNA agent is around 19-30 nucleotides in length.

10. The method of claim 9, wherein said siRNA is 21-23 nucleotides in length.

11. The method of claim 9, wherein said siRNA is double stranded.

12. The method of claim 1, wherein the agent is an antagonist antibody that specifically binds to the transmembrane receptor Ptch.

13. The method of claim 12, wherein the antibody is a monoclonal antibody.

14. A method for treating or ameliorating lymphoma or myeloma in a subject, the method comprising administering to the subject a pharmaceutical composition comprising an effective amount of an agent which down-regulates hedgehog signaling pathway.

15. The method of claim 14, wherein the lymphoma or myeloma in the subject does not express Gli3.

16. The method of claim 14, wherein the agent is an organic compound that specifically inhibits the hedgehog signaling pathway.

17. The method of claim 16, wherein the compound is cyclopamine or forskolin.

18. The method of claim 14, wherein the agent is a nucleic acid agent that specifically antagonizes a hedgehog signaling pathway member.

19. The method of claim 18, wherein the hedgehog signaling pathway member is Smoothened (Smo), Suppressor of Fused (SuFu), or transcription factor Gli.

20. The method of claim 18, wherein the agent is selected from the group consisting of an short interfering RNA (siRNA), a short hairpin RNA (shRNA), a microRNA (miRNA), an anti-sense nucleic acid, and a complementary DNA (cDNA).

21. The method of claim 14, wherein the agent is an antagonist antibody that specifically binds to the transmembrane receptor Ptch.

22. The method of claim 21, wherein the antibody is a monoclonal antibody.
